(19) United States
(12) Patent Application Publication
(10) Pub. No.: US 2025/0058127 A1
(43) Pub. Date: Feb. 20, 2025

(54) TEMPORARY IMPLANTABLE INTRACARDIAC DEFIBRILLATOR APPARATUS AND SYSTEM

(71) Applicant: Thomas Vacek, Hampton, VA (US)

(72) Inventor: Thomas Vacek, Hampton, VA (US)

(21) Appl. No.: 18/234,053

(22) Filed: Aug. 15, 2023

(51) Int. Cl. A61N 1/372; A61N 1/378; A61N 1/39

(52) U.S. Cl. CPC A61N 1/37223; A61N 1/37254; A61N 1/37258; A61N 1/3787; A61N 1/3925; A61N 1/39622; A61N 1/3987

(57) ABSTRACT

An apparatus and system for temporary implantable intracardiac defibrillator is provided to monitor and deliver therapy to a patient. The device is entirely implanted subcutaneously with minimal surgical intrusion into the body of a patient, and wirelessly interacts with external devices. If an arrhythmia occurs to a patient, the implant device activates an alarm to verify that the patient is not responsive. If the patient is conscious and wants to avoid a painful shock, the patient has a short time window to activate a shock aversion request. If the arrhythmia continues and the patient still does not respond, the patient will get a treatment shock.

### BACKGROUND OF THE INVENTION

The present invention generally relates to a cardiac stimulation device, more particularly relates to a temporary subcutaneous intracardiac defibrillator that can serve as a temporary bridge until a permanent implantable intracardiac defibrillator can be implanted or other measure can be taken.

Implantable medical devices are commonly used today to monitor physiological parameters of a patient and deliver therapy to a patient. For patients with heart-related conditions, various medical devices can be implanted in a patient's body. Implantable cardiac devices are such devices that are well known in the art. They may take the form of implantable cardioverter defibrillator (ICD), pacemaker, or both.

A vest defibrillator is a wearable medical device designed to continuously monitor a person's heart rhythm and deliver a life-saving shock if a life-threatening arrhythmia occurs It is typically prescribed for patients at high risk of sudden cardiac arrest.

If a life-threatening arrhythmia occurs to a patient, the device sounds an alarm to verify that the patient is not responsive. If the patient is conscious, the patient has a short time window, less than one minute, to respond to the alarms by pressing a push button to stop the treatment. If the arrhythmia continues and the patient still does not respond, the patient will get a treatment shock through the garment electrodes.

The use of a vest defibrillator carries the risk of potential periods during which cardiac arrest may occur, particularly when the vest must be taken off for bathing or when it is not worn.

The present invention of temporary implantable intracardiac defibrillator solves the problem of being able to monitor and shock the heart without having to wear a vest. The monitoring nature of the apparatus could also offer other values like measurement of impedance of fluid for continuous heart failure management and guidance to avoid heart failure decompensation and hospitalization and to further optimize medical management.

The present invention is temporary in nature for those who are: waiting for an ICD; waiting for a heart transplant; waiting to see if their condition doesn't improve and they need an ICD; or waiting to get another ICD after an infection or an ICD malfunction.

The present invention comprises an implant device to monitor and deliver therapy, an external device to interact with the implant device, and inductive power supplier to recharge a rechargeable battery of the implant device. The implant device is plunged into the subcutaneous tissue for temporary protection of heart arrhythmia and removed when needed.

### SUMMARY OF THE INVENTION

In view of the foregoing background, the present invention offers an apparatus and system to monitor and treat a person's arrhythmia on a temporary basis.

The implant device would be implanted much like an event monitor. The implant device would be installed with a plunger just like a traditional loop recorder. The sensing electrode and stimulation electrode of the device are positioned near the heart, not in the heart.

The implant device comprises central processor module, memory module, power supply module, communication module, arrhythmia sensing module, electrical shock generator module, input output processing module, leads/electrodes, and peripherals. The implant device is entirely implanted subcutaneously with no leads/electrodes available externally.

The power source will be depleted as it provides energy to drive the system. Battery replacement for implantable devices often requires additional surgery and can cause many complications. Alternatively, rechargeable batteries allow for longer useful lifetimes. Inductive power systems convert electromagnetic energy into electrical current allowing for highly efficient wireless energy transfer. The power supply module of the implant device is recharged through inductive powering.

The central processor module controls overall tasks of the implant device. The module controls detecting a cardiac anomaly, taking curative measures, and recording of cardiac events or any other events. The external device can retrieve the recording of cardiac events to analyze the heart rhythm. The communication module provides such functionality.

The implant device receives a command from an external device to transmit requested data to the external device. This requires two-way communication. Bidirectional communication provides greatly improved utility and functionality of implantable devices. In addition to the transmission of event data, additional functionality such as configuration change or a system status inquiry is implemented with the bidirectional communication.

However, this also introduces potential security concerns. The method for communication is critical for the patient's safety and security. For example, an unauthorized person or device can make changes to the implanted device and alter their functionality. Patient privacy may be compromised if unauthorized personnels or devices are able to access and read logged cardiac events. Secure communication protocols are available for security and safety considerations.

Upon detection of an arrhythmia, the central processor unit raises event detection alert and sends a command to begin high voltage charging to the capacitor of the electrical shock generator module. A therapeutic electrical shock can be delivered to the heart when the capacitor is fully charged.

A permanent ICD is not allowed to change its course of treatment by a patient. Taking a curative measure upon a detection of an anomaly is pre-programmed. However, the present invention is allowed to change its course of action by the patient. The patient can stop the delivery of shock if he is able to stop. The shock therapy is delivered only when the patient is unable to stop or chooses not to stop.

The present invention provides input and output mechanisms to the patient for this purpose. The output mechanism could vibrate to let the patient know that you are about to receive a shock. In order to avoid the imminent shock, the patient could either push a button on the external device to send a wireless stop command to the implant device or make a bodily motion to stop the imminent shock. The bodily motion may be tapping a button implanted in the chest of the patient, jiggling the implant device, patting chest generating high amplitude vector, patting on the chest near the implant device in a certain sequence that the device could recognize, making a linear acceleration to trigger accelerometer sensor, or making a rotational or leaning motion to trigger gyroscope sensor. The bodily motion can be any action that can be detected by motion sensors of the implant device. The shock is delivered when the patient is unconscious or willing to take the painful shock to avert possible grave harm.

With the present invention, clinicians have a choice. either the vest defibrillator or the present invention of the temporary implantable. Many people wear the vest for a few weeks or a few months. However, some people have worn the vest defibrillator for as long as several years.

### BRIEF DESCRIPTION OF DRAWINGS

FIG. 1 illustrates an embodiment of the present invention with an implant device subcutaneously implanted in a patient's body in relation to the patient's heart and an external device and an inductive power supplier.

FIG. 2 is another embodiment with multiple implant devices implanted in a patient.

FIG. 3 is a diagram of lead/electrode interface of an implant device.

FIG. 4 is a block diagram of the implant device.

FIG. 5 illustrates an embodiment of authentication between an implant device and an external device

FIG. 6 illustrates charging a rechargeable battery of an implant device with an inductive power supplier.

FIG. 7 is a logic flow diagram relating to detection of a cardiac anomaly, and delivery or aversion of a defibrillation.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiment set forth herein. Rather, these embodiments are provided so that this disclosure will be thorough and complete, and will fully convey the scope of the invention to those skilled in the art.

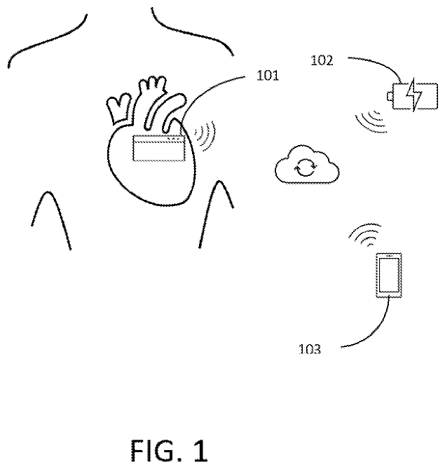

FIG. 1 illustrates a first embodiment of the present invention, having a single enclosure implanted in a patient. The implant device 101 is subcutaneously implanted near the patient's heart. The external device 103 wirelessly interacts with the implant device 101 and the inductive power supplier 102.

The external device 103 may be a computer. a mobile smartphone with suitable applications loaded, or a special purpose computing device with wireless communication capability.

Further referring to FIG. 1, bi-directional wireless links are shown allowing communication between the implant device and the external device. The wireless link is formed by a wireless protocol known in the art. Further, wireless energy transmission between the implant device and the inductive power supplier is implemented using inductive power transmission known in the art.

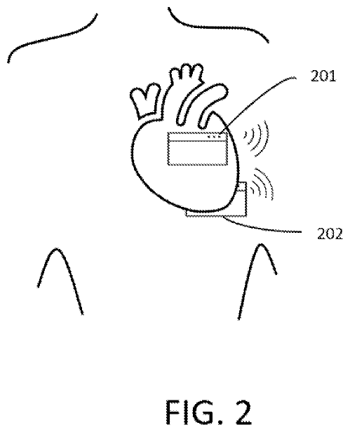

FIG. 2 illustrates a second embodiment of the present invention, having multiple enclosures implanted in a patient. The implant device 201 wirelessly interacts with another implant device 202. The functionality of the intracardiac defibrillator is divided into multiple enclosures.

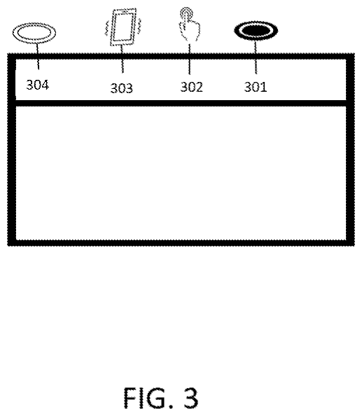

FIG. 3 is a diagram of peripheral devices connected to the electrodes of an implant device. The illustration includes an enclosure and leads interface of the implant device. Heart rhythm sensing leads are connected to the electrode 304. A vibrator is connected to the electrode 303. A bodily motion detection device is connected to the electrode 302. Electrical shock delivery leads are connected to the electrode 301. The heart rhythm sensing leads and electrical shock delivery leads are positioned at locations where the efficiency is maximized. The vibrator is positioned at a location in which its activation is sufficiently felt by the patient.

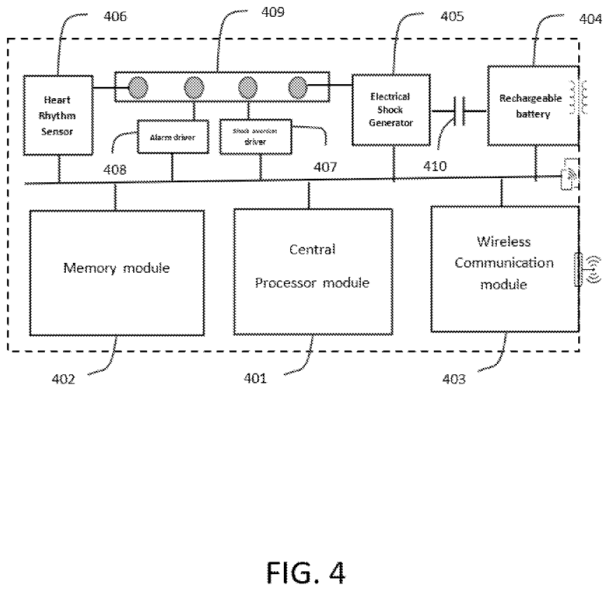

In FIG. 4, the implant device is a compact device with all components housed within one enclosure. The enclosure comprises a central processor module 401, a memory module 402, a wireless communication module 403, a rechargeable battery 404. an electrical shock generator module 405, a heart rhythm sensor module 406. a shock aversion driver 407, an alarm driver 408. I/O port 409, and capacitor 410.

The memory module 402 comprises a program section where instructions for the operations of the implant device are stored, and a data section where event data collected during the operation are temporarily stored. The central processor module 401 executes the instructions stored in the program section of the memory module 402. The central processor module controls the operation of the implant device.

The program running in the implant device can be updated. Such an updatability allows a user to modify the control logic or configuration data of the device even after implantation, thereby allowing for greater flexibility than when using a pre-programmed one.

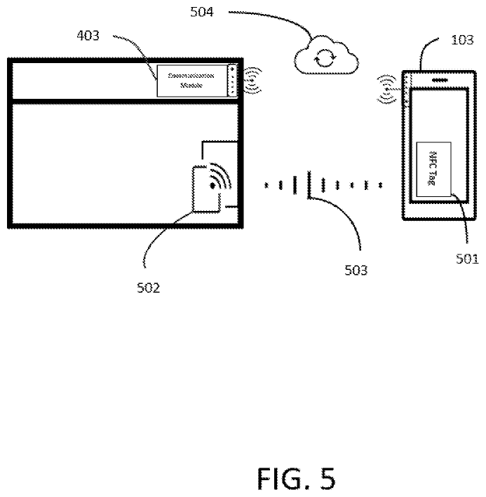

In FIG. 5, the communication module 403 of the implant device is configured to communicate with the external device 103. The communication between the module 403 and the device 103 comprises alerting cardiac event. responding to a request to stop the electrical shock, responding to system status inquiry, uploading stored logged event to the external device, and responding to configuration update request. Upon a detection of low power level on the rechargeable battery, the communication module 403 sends a charge request to the external device 103. The system status comprises whether the implant device is operating properly. whether communication is operating properly, whether there was any attempt to break into the implant device, and any other indication related to the operation of the devices.

The communication module may be configured to use one or more methods for communicating with external devices For example, the communication module may communicate via radiofrequency signals. or any other wireless mechanisms suitable for communication. The authentication between communication parties is critical for the patient's safety and security. In FIG. 5. an authentication embodiment of an NFC reader 502 reading an NFC tag 501 is shown as in 503. The communication channel 504 is open after successful authentication.

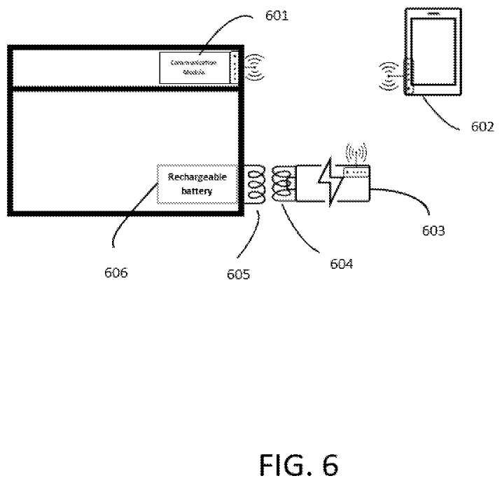

The power source is a battery that is enclosed within the housing of the device. The battery may be rechargeable or non-rechargeable. Because the device is an implantable device, access to the device may be limited after implantation. Accordingly, it is desirable to have sufficient battery capacity to deliver therapy over a period of treatment or the battery may be recharged, which may help increase the usable lifespan of the device. In FIG. 6, the implant communication module 601 sends a recharge request to the external device 602 upon a detection of low level of power remaining on the rechargeable battery 606. A patient or a clinician can initiate the recharging by placing the inductive power supplier 603 near the implant battery 606. Upon an established inductive coupling between the coil 604 and the coil 605, the external device 602 sends a charge command to start recharging the battery 606.

The electrical shock generator module 405 is electrically connected to an electrode of the I/O port 409. The electrical shock generator module is configured to generate electrical stimulation signals. The electrical shock generator includes one or more capacitors 410. The electrical shock generator charges the capacitors by drawing energy from the battery 404. The electrical shock generator may then use the energy of the charged capacitors to deliver the electrical stimulation via the electrode on the I/O port 409.

The electrical shock generator module may generate and deliver electrical stimulation signals with particular features or in particular sequences in order to provide one or multiple of a number of different stimulation therapies. A controller of the electrical shock generator may control the pulse voltage, pulse width, pulse shape or morphology, and/or any other suitable pulse characteristic.

In alternative embodiments, the electrical shock generator may include switching circuitry to selectively connect one or more of the electrodes to the electrical shock generator in order to select which of the electrodes the electrical shock generator delivers the electrical stimulation therapy to.

The heart rhythm sensor module 406 is configured to sense the cardiac electrical activity of the heart. The module is configured to receive cardiac electrical signals conducted through the electrodes of I/O port 409. The cardiac electrical signals may represent local information from the chamber in which the electrode is placed. The received signal information is used to determine whether an anomaly is occurring, a type of anomaly, and/or to take particular action in response to the information received. The sensor module is able to differentiate noise/artifact from ventricular fibrillation or ventricular tachycardia.

The alarm driver module 408 notifies a patient of an imminent electrical shock upon a detection of an abnormal heart rhythm requiring shock therapy. The alarm is activated to give the patient an option to avoid electrical shock. An electrically actuated vibrator connected to the alarm driver raises the alarm.

A patient may stop an imminent electrical shock by making a bodily motion to abort the shock. The shock aversion driver 407 comprises bodily motion detection sensors and circuitry to stop the shock delivery. Intentional activation of the shock aversion mechanism during a short period of time after the alarm will affect the operation of the implant device. Accidental activation of the aversion mechanism at any other time does not affect the operation of the implant device. A patient would know if the attempt to avert is recognized because the vibrator alarm would stop vibrating if the attempt is successful.

The electrodes on the I/O port 409 are disposed on the sides of the enclosure. The electrodes are connected to peripherals such as a bodily motion detection sensor, a vibrator, or leads to sense the heart rhythm or deliver the electrical shock to the surrounding tissue.

The operation of the invention is divided into therapy task and maintenance task. The therapy task comprises monitoring the heart rhythm signals from the heart rhythm sensor module 406, activating the alarm driver 408 when an abnormality is detected, choosing a course of therapy based on whether the shock aversion driver 407 is activated by a bodily motion or activation of the button on the external device 103, and activating the electrical shock generator 405 if instructed. Maintenance task comprises monitoring power level of the rechargeable battery 404, request and execution of the inductive charging of the battery 404, operation status inquiry and response, configuration change request and response, uploading logged clinical or event data to the external device, request to offload the logged data when the implant device is running out of data storage space, and logging authentication attempts to access the implant device.

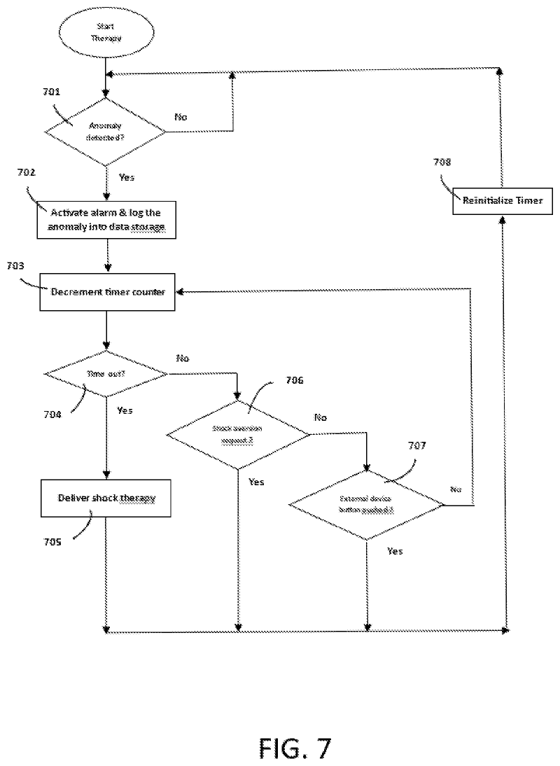

The flow chart FIG. 7 describes the logical flow of the therapy task. The task 701 receives and analyzes electrical signals coming from the heart rhythm sensor 406 in real time Once one of the monitored anomalies is detected, the task 702 activates an alarm by actuating the vibrator 303. The task 702 also logs the anomaly as an incident in the data storage space of the implant device. The task 703 counts down a timer whose value is initialized with a time period during which a patient can avoid an electrical shock either by performing the task 706 of activating the shock aversion request, or by performing the task 707 of pushing a button on the external device 103. The task 705 delivers electrical shock when the time runs out. It returns to the task 701 after reinitializing the timer at the task 708. The task 706 or the task 707 of averting the shock also returns to the task 701 after the timer reinitialization step of the task 708.

One skilled in the art, after being exposed to the teachings provided in the preceding descriptions and associated drawings, will likely conceive various modifications and alternative embodiments of the invention. Hence, it is important to note that the invention is not limited to the disclosed specific embodiments, and that modifications and alternative embodiments are intended to be encompassed within the scope of the appended claims.

Thus, the scope of the present disclosure is to be determined by the broadest permissible interpretation to the maximum extent allowed by law, of the following claims, and shall not be restricted or limited by the foregoing description.

1. In a system for monitoring heart conditions of a living body and providing therapy regimens to the body comprising two or more discrete devices, at least one of which is implanted into the living body, at least one of which is external to the living body, comprising:
implant device for monitoring and delivering therapy;
external device for providing inductive power to the implant device;
external device for interacting with the implant device and with the external device for providing inductive power to the implant device;
means for authentication between devices;
means for bi-directional wireless communication between devices; and
means for inductive power transfer through inductive coupling between the implant device and the inductive power supplier located external to the living body.

2. The implant device of claim 1 is a programming device comprising central processor module, memory module, communication module, battery module, heart rhythm sensor module, electrical shock generator module, shock aversion driver module, alarm driver module, input/output port, capacitor, electrodes, and peripherals, housed in one or more enclosures.

3. The implant device of claim 1 is implanted entirely subcutaneously with no lead available external to the body.

4. The programming device of claim 2 comprises means for:
receiving electrical signals appearing at the tissue-electrode interface of a living body;
detecting arrhythmia condition from the received electrical signals;
generating defibrillation shocks from said electrical shock generator module;
delivering defibrillation shocks to electrode placed in contact with a patient's heart;
logging the arrhythmia event in said memory module;
raising alert by activating a vibrator connected to the alarm driver module;
determining a course of action based on action or non-action of a patient; and
communicating with external devices regarding alerting cardiac event, providing status of the implant device. uploading logged event, updating configuration of the implant device, directing recharge of the battery, stopping the delivery of an electrical shock, and operation status inquiry and response.

5. The means for determining a course of action based on action or non-action of a patient of claim 4 comprises steps of:
the alarm drive module activating a vibrator to notify an imminent electrical shock when an anomaly is detected by the heart rhythm sensor module; and
counting down a timer to deliver a shock if neither a bodily motion triggering shock aversion driver nor pushing a shock aversion button on the external device is taken by the patient when the timer runs out.

6. The external device for interacting with the implant device and with external device for providing inductive power to the implant device of claim 1 is a programming device interacting with the implant device, and directing the inductive power supplier to charge the rechargeable battery of the implant device.

7. The external device for providing inductive power to the implant device of claim 1 starts charging the rechargeable battery of the implant device at the direction of the external device for interacting with the implant device and with external device for providing inductive power to the implant device of claim 1.

8. The means for authentication between devices of claim 1, wherein authentication must be successfully completed before a communication channel is opened between devices.

9. The means for bi-directional wireless communication between devices of claim 1 wherein, bi-directional wireless communication is accommodated between external devices, between implant devices, and between implant devices and external devices.

10. The means for inductive power transfer of claim 1, wherein the inductive power supplier is placed near the implant device to establish inductive coupling.
